(19) United States
(12) Patent Application Publication
(10) Pub. No.: US 2007/0237707 A1
(43) Pub. Date: Oct. 11, 2007

(54) System and method for generation and delivery of a biocidal agent

(76) Inventors: Ramanath Bhat, Billerica, MA (US); Nayantara Bhat, Billerica, MA (US)

Correspondence Address: BROMBERG & SUNSTEIN LLP, 125 SUMMER STREET, BOSTON, MA, 02110-1618, US

(21) Appl. No.: 11/238,836

(22) Filed: Sep. 29, 2005

(51) Int. Cl. C01B 11/02; B01J 8/04

(52) U.S. Cl. 423/477; 422/190; 422/222; 422/28

(57) ABSTRACT

An apparatus for generating a biocidal agent in a gaseous state. The apparatus includes a first monolith and second monolith. The first monolith includes a surface impregnated with an acid, while the second monolith includes a surface impregnated with a compound. The apparatus further includes a means for directing a gas that includes moisture sequentially to the first monolith and then to the second monolith. A gaseous stream that includes moisture having passed through the first monolith carries an acid vapor desorbed from the surface of the first monolith to the compound of the second monolith, whereby a stream of biocidal agent in a gaseous state is generated.

### TECHNICAL FIELD

The present invention relates to devices and methods for the generation of a biocidal agent, and more particularly to the generation and controlled delivery of a biocidal agent in a gaseous state to an enclosed environment so as to control biological agents and/or toxins.

### BACKGROUND ART

The use of toxic bio-organisms as weapons of mass destruction or terror is a major issue threatening the safety of a society. In this context, the solutions for combating water contamination is relatively more developed than systems that deal with contamination of the air with biotoxins. The use of molecular chlorine or chlorine dioxide solutions in many town water supply systems already provides a certain amount of protection against such water-borne biotoxins. The situation regarding air contamination is however, different. The magnitude of the damage done by deliberate acts of air contamination depends on whether the contaminant enters the open-air environment or a closed circuit air supply system.

Enclosed environments with a centralized supply of air offer an easy target for causing a major tragedy. An average urban dweller spends a significant part of his/her life in enclosed environments. Apartment buildings, offices, shopping centers and malls, churches. etc., use centralized air supply, heating and/or air conditioning systems. Lethal doses of biotoxins introduced into such systems can cause death and tragedy for thousands of people at a time. Accidental or deliberate contamination of the air supplied by a central air supply system to a large enclosed volume of space (like buildings, shopping malls, cinemas, subways etc.) with toxic bio-organisms, like anthrax, can lead to devastating consequences. Passive countermeasures that provide an effective and immediate response system triggered preferably by automatic detection and remedial measures for such contingencies would be advantageous.

Free molecular chlorine dioxide is an effective agent for the control of microorganisms and biological deposits. Gaseous chlorine dioxide in low concentrations up to 1000 ppm has long been known as useful for the treatment of odors and microbes. The use of gaseous chlorine dioxide is particularly advantageous where microbes or organic odorants are sought to be controlled. Chlorine dioxide functions without the formation of undesirable side products such as chloramines or chlorinated organic compounds that can be produced when elemental chlorine is utilized for the same or similar purposes. In addition, chlorine dioxide is also generally considered to be safe for human contacts at low concentrations that are effective for anti microbial applications. Generally, chlorine dioxide gas can be toxic to human beings only at concentrations greater than 100 ppm.

Chlorine dioxide can be explosive at pressures of 0.1 atmospheres and above. Consequently, it typically is not manufactured and shipped under pressure like other industrial gases. Rather, chlorine dioxide is often manufactured on-site. Conventional on-site manufacturing methods typically require not only expensive generation equipment but also high operator skill to avoid generating dangerously high concentrations.

More specifically, conventional methods that generate chlorine dioxide in a gaseous state on-site to large enclosed spaces have several drawbacks. For example, one known method of generating chlorine dioxide gas is to mix beds of zeolite crystals, where the first bed comprises a zeolite that has been impregnated with an aqueous solution of sodium chlorite, and the second bed comprises a zeolite that has been impregnated with phosphoric, citric or acetic acid. Chlorine dioxide gas is released when acid migrates from the second bed and contacts chlorite on the first bed. The first and second beds must be physically and intimately mixed together so that acid molecules desorbing from the first group of zeolite particles can react with the metal chlorite contained in the pores of the second group of the zeolite. This process requires expensive equipment and zeolite material. Moreover, due to the acidic and corrosive nature of the gaseous components involved, the zeolitic materials have a short shelf life. Another conventional method generates chlorine dioxide gas from a mixture of metal chlorite and a dry solid hydrophilic material such as meta-kaolin microspheres, on contact with liquid water or water vapor. One disadvantage of this process is that it generally requires a substantial humidification of the carrier gas stream. Other methods for producing chlorine dioxide gas involve complex on-site preparation of aqueous solutions involving long columns of liquids that are not suitable for decontamination of airspace in a large enclosed environment.

For fast and effective decontamination of a large enclosed airspace, such as in a buildings or subways, an advantageous solution is one that takes advantage of the existing air supply system to supply the chlorine dioxide. To efficiently supply large volumes of air into the enclosed volumes of space mentioned above, the biocidal agent generation system should impose a very low-pressure drop on the flow of the air stream. Hence, mixed beds of materials that include pellets or microspheres, or long columns of liquids for generating the anti toxins, are disadvantageous since such systems cause a large pressure drop, thereby increasing significantly the energy required for implementing such a biocide agent generation system.

There exists a need for controlled, on-site generation and delivery of required quantities of chlorine dioxide to large enclosed environments. Such a method and apparatus should produce the chlorine dioxide safely, efficiently and economically, without the necessity for a separate generation plant or a high-pressure compressor to overcome large pressure drops.

### SUMMARY OF THE INVENTION

In accordance with one embodiment of the invention an apparatus for generating a biocidal agent in a gaseous state includes a first monolith and second monolith. The first monolith includes a surface impregnated with an acid, while the second monolith includes a surface impregnated with a compound. The apparatus further includes a means for directing a gas that includes moisture sequentially to the first monolith and then to the second monolith. A gaseous stream that includes moisture having passed through the first monolith carries an acid vapor desorbed from the surface of the first monolith to the compound of the second monolith, whereby a stream of biocidal agent in a gaseous state is generated.

In accordance with related embodiments of the invention, the apparatus may be contained in a housing. The apparatus may be portable. The apparatus may include a saturator for adding moisture to the gas prior to passing the gas through the first monolith. The apparatus may include at least one of a scrubber and a filter. The apparatus may include a compressor, a power source and/or a sensor for detecting a biological agent and/or toxin. A controller may control flow of the gas through the first and second monoliths. The controller may be capable of varying the flow of gas through the first and second monoliths to obtain a predetermined concentration of biocidal agent. The apparatus may include one or more switches to control flow of gas through the first and second monoliths, wherein the controller controls the switches based on signals received from the sensor.

In accordance with another embodiment of the invention, a method of generating a biocidal agent in a gaseous state includes directing a gas that includes moisture to a first monolith having a surface impregnated with an acid, so as to generate an acid vapor stream having an acid vapor desorbed from the surface of the first monolith. The acid vapor stream is directed from the first monolith to a second monolith having a surface impregnated with a compound, so as to generate a stream of biocidal agent in a gaseous state.

In accordance with related embodiments of the invention, moisture may be added to the gas prior to directing the gas to the first monolith. The stream of biocidal agent may be directed to an enclosed environment, such as a building, a vehicle, and a tunnel. The vehicle may be an airplane, a ship, a truck, a bus or a train. The first and second monoliths may be contained within the enclosed environment. The flow rate of the gas may be controlled to vary the concentration of the biocidal agent delivered to the enclosed environment. Directing the gas to the first monolith may be based on detecting a toxin and/or biological agent in the enclosed environment. Directing the gas to the first monolith upon detection of the toxin and/or biological agent may either be an automatic process or a manual process. Directing of the gas to the first monolith may include directing air from either outside the enclosed environment or from the enclosed environment.

In accordance with another embodiment of the invention, a container for generating a biocidal agent includes an inlet and an outlet. At least one monolith is disposed within the container. The at least one monolith is impregnated with one of an acid and a compound.

In accordance with related embodiments of the invention, the container may include opening means such that at least one monolith can be removed and/or inserted. After removal, the at least one monolith may be capable of being replaced with an acid and a compound, respectively. The at least one monolith may include a first monolith and a second monolith. The first monolith may be disposed adjacent the inlet, and include a surface impregnated with an acid. The second monolith may be disposed adjacent the first monolith, the second monolith including a surface impregnated with a compound. The outlet may be adjacent the second monolith.

In accordance with another embodiment of the invention, a biocidal agent delivery system for an enclosed environment includes a first monolith and a second monolith. The first monolith includes a surface impregnated with an acid, while the second monolith includes a surface impregnated with a compound. A duct system directs a gas that includes moisture sequentially to the first monolith and then to the second monolith. A gaseous stream that includes moisture having passed through the first monolith carries an acid vapor desorbed from the surface of the first monolith to the compound of the second monolith, whereby a stream of biocidal agent is generated and delivered via the duct system to the enclosed environment.

In accordance with related embodiments of the invention, the biocidal agent delivery system may further include a saturator for adding moisture to the gas prior to passing the gas through the first monolith. The gas inlet system may include a switch, the switch capable of bypassing the flow of the gas to the first monolith when in a first position, and permitting the flow of the gas to the first monolith when in a second position. The switch in the second position may direct air from the enclosed environment to the first monolith from one of an existing HVAC system associated with the enclosed environment and/or an independent gas delivery system. A sensor may be used to detect a toxin and/or a biological agent in the enclosed environment. A controller may move the switch to the second position upon the sensor detecting the toxin and/or the biological agent. A controller may control the flow rate of the gas, such that the concentration of biocidal agent delivered to the enclosed environment can be varied.

In accordance with further related embodiments of the invention, the first and second monoliths may be contained within the enclosed environment. The enclosed environment may be one of a building, a vehicle and a tunnel. The vehicle may be, for example, an airplane, a ship, a truck, a bus or a train.

In accordance with another embodiment of the invention, a monolith for use in a canister for generating biocidal agent in a gaseous state is presented. The canister includes an inlet and an outlet. The monolith includes channels for facilitating flow of a gas, and a surface impregnated with one of an acid and a compound.

In accordance with embodiments related to the above-described embodiments of the invention, the acid impregnated on the first monolith may be a sulfuric acid, a hydrochloric acid, a nitric acid, a propionic acid, an acetic acid, and a fluorosulphonic acid. The biocidal agent may be chlorine dioxide. The compound impregnated on the second monolith used in forming the chlorine dioxide may be a metal chlorite. The biocidal agent may be ethylene oxide. The compound used in forming the ethylene oxide may be ethylene glycol. The first monolith and/or the second monolith may be made of a metal, an iron alloy, a nickel alloy, an aluminum alloy, a ceramic, a cordierite or any other material that can act as a support. The surface of the monolith(s) may be substantially dry.

In accordance with another embodiment of the invention, a method of treating an enclosed space with chlorine dioxide includes passing a gas that includes moisture to an apparatus. The apparatus includes a first monolith and a second monolith. The first monolith includes a surface impregnated with an acid, while the second monolith includes a surface impregnated with a metal chlorite. The apparatus further includes means for directing the gas that includes moisture sequentially to the first monolith and then to the second monolith. A gaseous stream that includes moisture having passed through the first monolith carries an acid vapor desorbed from the acid surface of the first monolith to the metal chlorite of the second monolith, whereby a stream of chlorine dioxide is generated. The stream of chlorine dioxide is directed to the enclosed space to be treated with chlorine dioxide.

In accordance with another embodiment of the invention, a method of generating chlorine dioxide includes passing a gas that includes moisture through a first stage including a monolith material, wherein the said monolith material includes an alumina washcoat impregnated with an acid. The effluents containing the vapors of the said acid are passed through a second stage including a second monolith washcoated with alumina on which sodium chlorite has been impregnated. The flow rate of the gas is controlled so that the concentration of chlorine dioxide produced by the reaction of the acid vapors contained in the effluent from the first stage with the sodium chlorite contained in the washcoat of the second stage, does not exceed substantially a percentage the weight of the gaseous stream of air. The percentage may be, without limitation, between 1%-15%.

### BRIEF DESCRIPTION OF THE DRAWINGS

The foregoing features of the invention will be more readily understood by reference to the following detailed description, taken with reference to the accompanying drawings, in which:

FIG. 1 is a schematic of a biocidal agent delivery system, in accordance with one embodiment of the invention;

FIG. 2 is a schematic showing an exemplary monolith, in accordance with one embodiment of the invention;

FIG. 3 is a flowchart for a method of delivering a biocidal agent, in accordance with one embodiment of the invention; and

FIG. 4 is a schematic of a biocidal agent delivery system, in accordance with one embodiment of the invention.

### DETAILED DESCRIPTION OF SPECIFIC EMBODIMENTS

In illustrative embodiments of the invention, there is provided a novel method and apparatus for the generation and delivery of a biocidal agent, such as chlorine dioxide gas, into an enclosed environment. The unique generation-cum-delivery method and apparatus is generally based on a pair of alumina washcoated monoliths with active ingredients that control the rate and efficiency of generation of the biocidal agent and distribute it uniformly into a large volume of air. Various examples and embodiments are discussed in detail below.

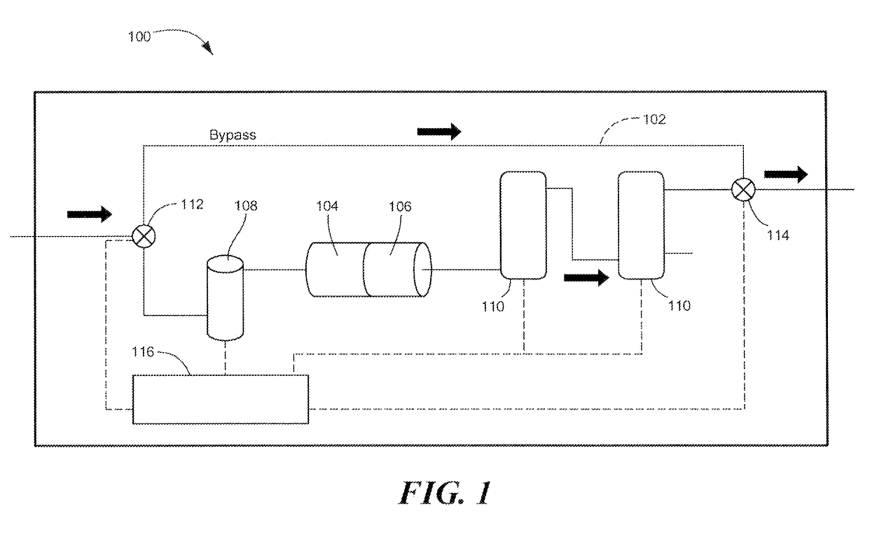

FIG. 1 is a schematic of a biocidal agent delivery system 100, in accordance with one embodiment of the invention. The system 100 may include ducts 102 for directing gas through the system 100 and further to the enclosed environment. In various embodiments, the ducts 102 may be part of an existing HVAC system of a building, or alternatively, an independent gas delivery system. Check valves may be positioned at various locations in the flow path to prevent backflow.

The biocidal agent may be delivered, without limitation, to an enclosed environment. The enclosed environment may be, for example, a building, a vehicle, and a tunnel. The vehicle may be, without limitation, an airplane, a ship, a truck, a bus or a train.

The size of the enclosed environment may be, without limitation, such that the throughput of air required through the system 100 is very large (air flow of thousands of cubic meters of air per minute). Hence, in order to minimize the energy costs, the pressure drop across the system may advantageously be made minimal.

Accordingly, the system 100 utilizes one or more monoliths 104 and 106 for contacting large volumes of gas with a large surface area of a solid with minimal pressure drops. As used in this description and the accompanying claims, the term “monolith” shall mean, unless the context otherwise requires, a block or element made from a single piece of material. A plurality of materials joined to substantially act as a single piece of material is also to be considered a monolith. The monoliths 104 and 106 may be made of, without limitation, a metal, an iron alloy, a nickel alloy, an aluminum alloy, a ceramic, a cordierite and/or any other material that can act as a support. The monoliths 104 and 106 may vary in size and/or shape. For example, the monolith may be circular or rectangular in nature.

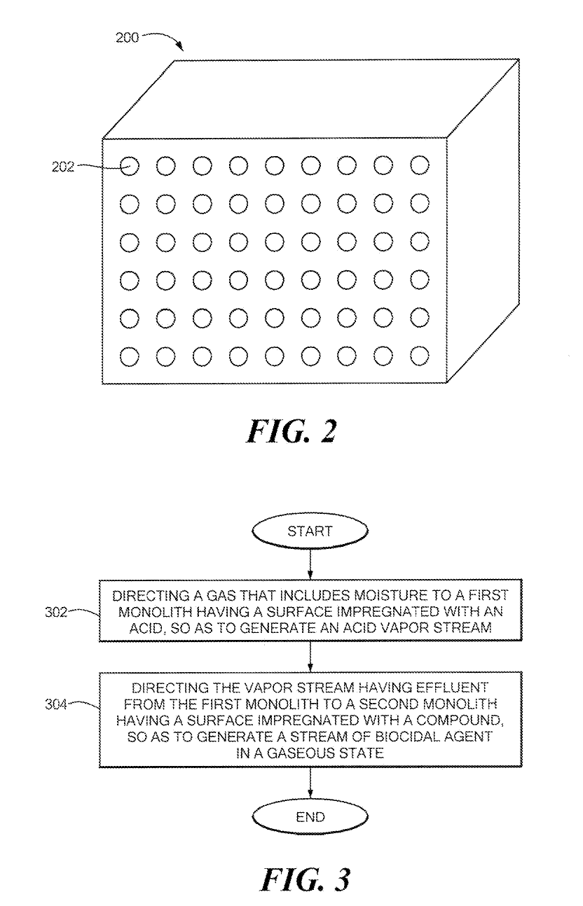

FIG. 2 is a schematic showing an exemplary monolith 200, in accordance with one embodiment of the invention. The gas flow through the monolith 200 may be facilitated by the presence of channels 202, which run across the entire length of the monolith 200 from the inlet to the outlet of the monolith 200. Across a given cross section of the monolith 200, there may be channel openings ranging, without limitation, from 10 to as high as 800 openings per square inch. Such monoliths are available commercially. In various embodiments, the pressure drop across the monolith 200 may increase with the number of channels per square inch, for example, where the diameter of the channel openings of the monolith 200 decreases with a higher number of channels.

In various embodiments, the monoliths 104 and 106 shown in FIG. 1 may be contained within the enclosed environment. Generally, the monoliths are chemically inactive when not in use, and represent minimal risk even when in use. The monoliths are furthermore sized to be practically placed within enclosed environments such as a building. As discussed above, the monoliths may advantageously be part of the existing HVAC system of the enclosed environment.

The one or more monoliths may be enclosed in at least one canister, which may be attached, for example, to the duct system 102. While the monoliths may be stored in the same canister, in other embodiments each monolith may be held in a separate canister to provide further isolation and inadvertent passage of gas between the monoliths. The canister may include an inlet and an outlet. In embodiments in which the canister holds two monoliths, the first monolith having a surface impregnated with acid may be positioned proximate the inlet, while a second monolith impregnated with a compound may be positioned serial to the first monolith and proximate the outlet. The two monoliths within the single canister may each be further held in separate containers to provide isolation between the monoliths. In preferred embodiments, the canister and/or containers may include opening means such that the one or more monoliths can be removed and/or inserted. The opening means may include, without limitation, a hinged panel, with a fastening or locking mechanism to keep the panel securely closed when not in use. When the opening means is closed, the canister may be substantially sealed, except for the inlet and outlet, to limit the escape of gas from within the canister. Upon removal, the first and/or second monolith may be capable of being replaced and subsequently placed back in the canister and/or container. Alternatively, the first and/or second monoliths may be reimpregnated in-situ with an acid and a compound, either automatically or manually.

In illustrative embodiments, the biocidal agent that the system 100 generates may be, without limitation, chlorine dioxide gas. In other embodiments, the system 100 may deliver a variety of other biocidal agents, such as ethylene oxide. A combination of biocidal agents may be generated, such as a combination of both ethylene oxide and chlorine dioxide. In preferred embodiments, the biocidal agent is in the form of a gas. The biocidal agent may be used to control or neutralize, without limitation, a variety of biological agents, toxins and/or molds. For example, the biocidal agent may effectively neutralize, without limitation, anthrax spores, avian flu virus, HIV/AIDS virus and/or various odors. The biocidal agent may be used to disinfect water, such as pool water or a drinking water supply.

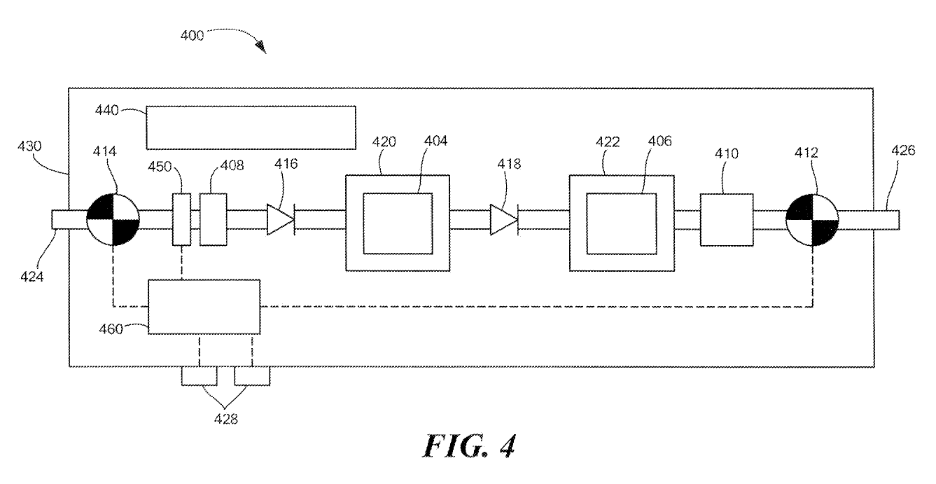

FIG. 3 is a flowchart for a method of delivering a biocidal agent, in accordance with one embodiment of the invention. A gas that includes moisture is directed to the first monolith 104 having a surface impregnated with an acid, so as to generate an acid vapor stream, step 302. The acid component, such as sulfuric acid, hydrochloric acid, nitric acid, propionic acid, acetic acid, or fluorosulphonic acid, may be deposited on an alumina washcoat of the first monolith 104. The alumina washcoat on the monolith 104 may comprise, without limitation, gamma alumina.

The vapor stream having effluents from the first monolith is then passed through a second monolith 106, step 304. The content of the acid component deposited on the first monolith 104 is chosen such that with normal flow rates of humid air through the monolith, the amount of acid desorbed is adequate to react with a compound deposited on the second monolith 106 to produce the desired biocidal agent in a gaseous state. On emerging from the outlet of the second monolith 106, the biocide agent is delivered into the enclosed environment.

For example, a metal chlorite may be deposited on the second monolith 106, such that an adequate quantity of chlorine dioxide is liberated. Instead of metal chlorite, other compounds may be used to produce different biocidal agents. The compound may be ethylene glycol, such that ethylene oxide is liberated. The concentration of the acid in the gaseous effluent from the first monolith 104 may be controlled, for example, by the amount of acid component deposited on the first monolith 104, and/or the flow rate of the stream of air passing through the monolith containing the acid component. In various embodiments, the air stream emerging from the outlet of the second monolith 106 may pass through one or more scrubbers and/or filters 110, such as activated carbon filters. The scrubbers and/or filters 110 may remove, without limitation, acid or other undesired components prior to the biocidal agent delivered into the enclosed environment.

The compound disposed within the pores of an alumina washcoat deposited on the surface of the second monolith 106 generates the desired biocidal agent in the presence of an initiating agent like the vapors of an acid. Illustratively, the initiating agent for the release of chlorine dioxide from sodium chlorite contained in the washcoat of the second monolith 106 is the above-described vapor of acid in the gaseous air stream from the first monolith 104. Acid components, like H₂SO₄, HCl, HNO₃, acetic acid, propionic acid and citric acid deposited on a first monolith 104 will desorb in a stream of incoming air and react with the sodium chlorite, NaClO₂, deposited on the second monolith 106 placed downstream of the first monolith 104. Subsequently, chlorine dioxide gas, ClO₂, will be liberated in concentrations sufficient to neutralize biological agents such as, without limitation, anthrax spores, avian flu virus, and/or HIV/AIDS virus, but not be harmful to human beings in the enclosed environment.

An exemplary generic chemical reaction with regard to the formation of the chlorine dioxide is:
NaClO₂+acid vapor→ClO₂+metal salt

The chemical stoichiometry of the reaction is illustrated with the example of the use of sulfuric acid as the acid for generating the ClO₂ from the NaClO₂ contained in the washcoat of the second monolith 106:
5NaClO₂+2.5H₂SO₄→4ClO₂+2.5Na2SO₄+HCl+2H₂O

The sulfuric acid vapor is generated from H₂SO₄ deposited on an alumina washcoat on the first monolith 104 placed in front of the second monolith 106 containing the NaClO₂.

By using discreet amounts of acid and sodium chlorite contained within the two monolith system 100, and varying the flow rate of the gaseous air stream flowing through them, a chemical reaction generating a specific concentration of chlorine dioxide gas may be generated from the NaClO₂.

For example, an embodiment of the present invention provides for a method of generating chlorine dioxide by passing a gaseous stream of air through a first stage including a monolith material, wherein the said monolith material includes an alumina washcoat impregnated with an acid. The effluents containing the vapors of the said acid are passed through a second stage including a second monolith washcoated with alumina on which sodium chlorite has been impregnated. The flow rate of the gaseous stream of air is controlled so that the concentration of chlorine dioxide produced by the reaction of the acid vapors contained in the effluent from the first stage with the sodium chlorite contained in the washcoat of the second stage, does not exceed substantially a percentage the weight of the gaseous stream of air. The percentage may be, without limitation, between 1%-15%

The above-described embodiments for generating chlorine dioxide can be modified in various different ways for specific applications. For example, the release of chlorine dioxide may be made sustainable even after the acid on the first monolith 104 and the sodium chlorite in the second monolith 106 are consumed. In various embodiments, biotoxin sensors may be installed in the enclosed space to be decontaminated that indicate the presence of biotoxins even after the concentration of the chlorine dioxide in the effluent from the exit of the second monolith fall below, for example, 1 ppm. This may trigger a controller to open a valve which can allow a stream of liquid acid, such as sulfuric acid, to pass into the first reaction zone containing the first monolith 104 from which all the acid had been consumed. The flow of liquid acid can be terminated once its concentration is detected at the exit of the first monolith 104. Similarly, and typically simultaneously with the start of flow of the liquid acid into the first reaction zone, the controller may trigger opening of another valve that allows a stream of saturated sodium chlorite to pass into the second reaction zone containing the second monolith from which all the sodium chlorite deposited earlier had been consumed. This flow of the saturated solution of sodium chlorite can be terminated once its concentration in significant quantities is detected in the effluent from the second reaction zone.

In various embodiments of the invention, the system 100 may include a saturator 108 for adding moisture to the gas prior to passing the gas through the first monolith 104. For example, the humidity of the incoming air stream may be inadequate (e.g., below 30%) to desorb the acid from the first monolith 104 and activate the reaction with the second monolith 106 to generate the chlorine dioxide. Water sprinklers or bubblers 108 may then be activated manually or automatically based, for example, on readings from a humidity sensor positioned in the incoming gas stream prior to the first monolith 104. The humidity of the gas stream passing over the first monolith can be increased using the saturator 108 to levels adequate (e.g., above 30%) enough to desorb the acid from the first monolith 104 and consequently activate the reaction and generate the desired amount of chlorine dioxide from the second monolith 106.

The monoliths may be pre-treated with a mild alkali solution of pH=7-8 before washcoating. This may be done, for example, to prevent the material of the first and/or second monoliths 104 and 106 from reacting with the acid deposited on the first monolith 104. Examples of such alkali solutions include sodium hydroxide and potassium hydroxide. In various embodiments, the second monolith may be pre-treated with a mild alkali solution of pH=7-8 before washcoating to prevent the material of the second monolith from reacting with, for example, the NaClO₂ deposited on the second monolith. Examples of such alkali solutions include sodium hydroxide and potassium hydroxide. Depending on the application, the surfaces of the monolith may be dry or wet.

Fumigation of a building to kill all the biotoxins, such as anthrax spores, for example, will involve exposing all interiors of the building to chlorine dioxide. Normally the fumigation process should not exceed a couple of days. The chlorine dioxide generated as hereinabove described may be injected into the building at multiple locations to ensure even distribution of the gas throughout the building.

To avoid chlorine dioxide gas escaping the building during fumigation, negative pressure may be maintained throughout the building by continuous exhausting of air from the building through multiple control devices. High efficiency particulate air filters may capture any biotoxins that might come out with the air exhaust from the building. The chlorine dioxide gas produced may be passed over the filters to ensure that any toxins collected on the filters are no longer a threat.

As shown in FIG. 1, the biocidal agent delivery system 100 may include a controller 116 for controlling, without limitation, the flow of gas to the first monolith 104 and/or the discharge of the biocidal agent. To accomplish this, the duct system 102 may include one or more switches 112 and 114 that are controlled by the controller 116. Note that alternatively, these switches 112 and 114 may be controlled manually.

Switch 112 may be capable, for example, of bypassing the flow of the gas to the first monolith 104 when in a first position, and permitting the flow of the gas to the first monolith 104 when in a second position. In the second position, air flow from the existing HVAC associated with the enclosed environment may be passed through the biocidal agent delivery system 100, or alternatively, an independent gas delivery system may deliver air flow to the first monolith 104, as described above. Switches 112 and 114 and other components in the system 100 may also be controlled by the controller 116 to vary the flow of gas through the monoliths, such that a desired concentration of biocidal agent is obtained. The controller 116 may receive signals from a flow meter placed in the flow path of the gas.

The system 100 may include one or more sensors (not shown) for sensing a biological agent and/or toxin within the closed environment. The controller may control the one or more switches 112 and 114 based upon the sensor detecting the biological agent and/or toxin. A sensor may also be used to detect the presence of the biocidal agent and/or toxin.

FIG. 4 shows a schematic of another biocidal agent delivery system 400, in accordance with one embodiment of the invention. The system 400 is enclosed in a housing 430. When in use, housing 430 may be substantially sealed to limit the escape of gas from within the housing 430, except via inlet 424 and outlet 426. The housing 430 may vary in size according to application. In various embodiments, the housing 430 is sized to be handheld and/or portable. For example, the housing 430 may be roughly the size of a flashlight. The housing may be capable of being opened via, without limitation, a pivoting door. The system 400 may includes valves 412 and 414 to control the flow of gas at inlet 424 and outlet 426. The system 400 may include a compressor 450 for pumping gas from inlet 424 through system 400 and outlet 426. A power supply 440, such as a battery, may supply power to one or more components within the system 400. In other embodiments, the system 400 may be supplied with power via, for example, a wall socket.

Similar to above-described embodiments, check valves 416 and 418 may be positioned at various locations in the flow path to prevent backflow. The system includes one or more monoliths 404 and 406. A first monolith 404 may be positioned in a first container 420, with a second monolith 406 positioned in a second container 422 to further isolate the monoliths 404 and 406 when the system 400 is not in use. The first monolith 404 and/or the second monolith 406 may be capable of being removed from the first container 420 and second container 422, respectively, such that the first monolith 404 and/or the second monolith 406 can be replaced and/or recharged. The system 400 may further include a saturator 408 for adding moisture to the gas prior to passing through the first monolith 404 and the second monolith 406. A controller 460 may be used to control, without limitation, the valves 412 and 414 and the compressor 450. The controller 460 may receive signals from one or more sensors 428 that detect the presence of a biotoxin and/or the biocidal agent. Upon detection of a biotoxin, the controller 460 may automatically control valves 412 and 414 and/or compressor 450 to produce the biocidal agent. The system 400 may be hooked into an existing HVAC of a building. As described above, an acid may be deposited on the first monolith 404 and a compound may be deposited on the second monolith 406, such that gas passing through the first and second monoliths 404 and 406 generates the biocidal agent.

The following example is illustrative of embodiments of the invention and is not intended to limit the invention as encompassed by the claims forming part of this application.

### EXAMPLE 1

A 4.5-inch diameter and 5-inch high cylindrical monolith made of cordierite and on which a gamma alumina washcoat had been deposited was soaked in a beaker containing 500 mL of 0.1 M HNO₃. After the uptake of nitric acid had been completed, the monolith was removed and dried at room temperature overnight. This monolith is referred to as the first monolith.

A second, similar monolith was soaked in 500 mL of an aqueous saturated solution of sodium chlorite at room temperature for 24 hours under stirring. The monolith was then taken out and dried at room temperature. This monolith is referred to as the second monolith.

The two monoliths were packed into a cylindrical S.S.316 metallic tube fitted with attachments for the inlet and outlet of air with the first monolith placed near the inlet and the second monolith placed near the outlet. Provisions were also made for the measurement of the flow of air through the inlet and outlet. The chlorine dioxide gas concentration in the outlet air stream was measured by Draeger R.T.M. model CH24301 tubes or commercially available detectors such as ToxiPro. Air was supplied by a laboratory air compressor. This air after passing through the gas flow regulators and flowmeters was passed directly to the inlet of the metallic tube containing the two monoliths.

At a gas hourly space velocity, GHSV, of 0.4 million (i.e. 0.4 million ml of air per ml of the volume of the monoliths), the concentration of chlorine dioxide in the outlet stream varied from 100 to 1 ppm over a period of 24 hours. This is considered adequate for the type of applications mentioned hereinabove.

Although various exemplary embodiments of the invention have been disclosed, it should be apparent to those skilled in the art that various changes and modifications can be made which will achieve some of the advantages of the invention without departing from the true scope of the invention. These and other obvious modifications are intended to be covered by the appended claims.

What is claimed is:

1. An apparatus for generating a biocidal agent in a gaseous state, the apparatus comprising:
a first monolith including a surface impregnated with an acid;
a second monolith including a surface impregnated with a compound; and
means for directing a gas that includes moisture sequentially to the first monolith and then to the second monolith, wherein a gaseous stream that includes moisture having passed through the first monolith carries an acid vapor desorbed from the surface of the first monolith to the compound of the second monolith, whereby a stream of biocidal agent in a gaseous state is generated.

2. The apparatus according to claim 1, wherein the biocidal agent is chlorine dioxide.

3. The apparatus according to claim 2, wherein the compound is a metal chlorite.

4. The apparatus according to claim 1, wherein the biocidal agent is ethylene oxide.

5. The apparatus according to claim 4, wherein the compound is ethylene glycol.

6. The apparatus according to claim 1, wherein the surface of at least one of the first and second monolith is substantially dry.

7. The apparatus according to claim 1, wherein at least one of the first monolith and the second monolith includes channels for facilitating flow of the gaseous fluid.

8. The apparatus according to claim 1, wherein at least one of the first monolith and the second monolith are made of a material chosen from the group consisting of a metal, an iron alloy, a nickel alloy, an aluminum alloy, a ceramic, and cordierite.

9. The apparatus according to claims 1, wherein the acid is selected from the group consisting of sulfuric acid, hydrochloric acid, nitric acid, propionic acid, acetic acid, and fluorosulphonic acid.

10. The apparatus according to claim 1, wherein the apparatus is contained in a housing.

11. The apparatus according to claim 10, wherein the apparatus is portable.

12. The apparatus according to claim 1, wherein the apparatus includes a saturator for adding moisture to the gas prior to passing the gas through the first monolith.

13. The apparatus according to claim 1, wherein the apparatus includes at least one of a scrubber and a filter.

14. The apparatus according to claim 1, wherein the apparatus includes a compressor.

15. The apparatus according to claim 1, wherein the apparatus includes a power source.

16. The apparatus according to claim 1, further comprising:
a sensor for detecting at least one of a biological agent and a toxin.

17. The apparatus according to claim 1, further comprising a controller for controlling flow of the gas through the first and second monoliths.

18. The apparatus according to claim 17, wherein the controller is capable of varying the flow of gas through the first and second monoliths to obtain a predetermined concentration of biocidal agent.

19. The apparatus according to claim 17, further comprising:
a sensor for detecting at least one of a biological agent, a toxin, and the biocidal agent; and
one or more switches to control flow of gas through the first and second monoliths, wherein the controller controls the switches based on signals received from the sensor.

20. A method of generating a biocidal agent in a gaseous state, the method comprising:
directing a gas that includes moisture to a first monolith having a surface impregnated with an acid, so as to generate an acid vapor stream having an acid vapor desorbed from the surface of the first monolith; and
directing the acid vapor stream from the first monolith to a second monolith having a surface impregnated with a compound, so as to generate a stream of biocidal agent in a gaseous state.

21. The method according to claim 20, wherein the biocidal agent is chlorine dioxide.

22. The method according to claim 21, wherein the compound is a metal chlorite.

23. The method according to claim 20, wherein the biocidal agent is ethylene oxide.

24. The method according to claim 23, wherein the compound is ethylene glycol.

25. The method according to claim 20, wherein the surface of at least one of the first and second monoliths is substantially dry.

26. The method according to claim 20, further comprising:
adding moisture to the gas prior to directing the gas to the first monolith.

27. The method according to claim 20, further comprising:
directing the stream of biocidal agent to an enclosed environment.

28. The method according to claim 27, wherein the enclosed environment is one of a building, a vehicle, and a tunnel.

29. The method according to claim 28, wherein the vehicle is selected from the group consisting of an airplane, a ship, a truck, a bus and a train.

30. The method according to claim 20, wherein the first and second monoliths are contained within the enclosed environment.

31. The method according to claim 27, further comprising detecting at least one of a toxin and a biological agent in the enclosed environment, wherein the directing of the gas to the first monolith is based on the detection of the at least one of a toxin and a biological agent.

32. The method according to claim 31, wherein the directing of the gas to the first monolith upon detection of the at least one of the biological agent and the toxin is one of an automatic process and a manual process.

33. The method according to claim 31, wherein the directing of the gas to the first monolith includes directing air from one of outside the enclosed environment and from the enclosed environment.

34. The method according to claim 30, further comprising varying the flow rate of the gas, so as to control the concentration of the biocidal agent delivered to the enclosed environment.

35. A container for generating biocidal agent comprising:
an inlet;
at least one monolith disposed within the container, the at least one monolith impregnated with one of an acid and a compound; and
an outlet.

36. The container according to claim 35, wherein the biocidal agent is chlorine dioxide.

37. The container according to claim 36, wherein the compound is a metal chlorite.

38. The container according to claim 35, wherein the biocidal agent is ethylene oxide.

39. The container according to claim 38, wherein the compound is ethylene glycol.

40. The container according to claim 35, wherein the at least one monolith includes a first monolith and a second monolith, the first monolith disposed adjacent the inlet, the first monolith including a surface impregnated with an acid, the second monolith disposed adjacent the first monolith, the second monolith including a surface impregnated with a compound, the second monolith disposed adjacent the outlet.

41. The container according to claim 35, wherein the canister includes opening means such that at least one monolith can be removed and/or inserted.

42. The container according to claim 41, wherein after removal the at least one monolith is capable of being one of replaced and reimpregnated with one of an acid and a compound.

43. The container according to claim 35, wherein the at least one monolith includes channels for facilitating flow of the gas.

44. The container according to claim 35, wherein the at least one monolith is made of a material chosen from the group consisting of a metal, an iron alloy, a nickel alloy, an aluminum alloy, a ceramic, and cordierite.

45. The container according to claims 35, wherein the acid is selected from the group consisting of sulfuric acid, hydrochloric acid, nitric acid, propionic acid, and fluorosulphonic acid.

46. A biocidal agent delivery system for an enclosed environment comprising:
a first monolith including a surface impregnated with an acid;
a second monolith including a surface impregnated with a compound;
a duct system for directing a gas that includes moisture sequentially to the first monolith and then to the second monolith, wherein a gaseous stream that includes moisture having passed through the first monolith carries an acid vapor desorbed from the surface of the first monolith to the compound of the second monolith, whereby a stream of biocidal agent is delivered via the duct system to the enclosed environment.

47. The biocidal agent delivery system according to claim 46, wherein the biocidal agent is chlorine dioxide.

48. The biocidal agent delivery system according to claim 47, wherein the compound is a metal chlorite.

49. The biocidal agent delivery system according to claim 46, wherein the biocidal agent is ethylene oxide.

50. The biocidal agent delivery system according to claim 49, wherein the compound is ethylene glycol.

51. The biocidal agent delivery system according to claim 46, wherein the surface of at least one of the first and second monolith is substantially dry.

52. The biocidal agent delivery system according to claim 46, further comprising a saturator for adding moisture to the gas prior to passing the gas through the first monolith.

53. The biocidal agent delivery system according to claim 46, wherein the duct system includes a switch, the switch capable of interrupting the flow of the gas to the first monolith when in a first position, and permitting the flow of the gas to the first monolith when in a second position.

54. The biocidal agent delivery system according to claim 53, wherein the switch in the second position directs air from the enclosed environment to the first monolith from one of an existing HVAC system associated with the enclosed environment and an independent gas delivery system.

55. The biocidal agent delivery system according to claim 53, further comprising:
a sensor for detecting at least one of a biological agent and a toxin in the enclosed environment.

56. The biocidal agent delivery system according to claim 55, further comprising a controller for switching the switch to the second position upon the sensor detecting the at least one of the biological agent and the toxin.

57. The biocidal agent delivery system according to claim 56, further comprising a controller for controlling the flow rate of the gas, such that the concentration of biocidal agent delivered to the enclosed environment can be varied.

58. The biocidal agent delivery system according to claims 56, wherein the acid is selected from the group consisting of sulfuric acid, hydrochloric acid, nitric acid, propionic acid, acetic acid and fluorosulphonic acid.

59. The biocidal agent delivery system according to claim 56, wherein the first and second monoliths are contained within the enclosed environment.

60. The biocidal agent delivery system according to claim 56, wherein the enclosed environment is one of a building, a vehicle and a tunnel.

61. The biocidal agent delivery system according to claim 60, wherein the vehicle is selected from the group consisting of an airplane, a ship, a truck, a bus and a train.

62. The biocidal agent delivery system according to claim 56, wherein at least one of the first monolith and the second monolith is made of a material chosen from the group consisting of a metal, an iron alloy, a nickel alloy, an aluminum alloy, a ceramic, and cordierite.

63. A method of treating an enclosed space with chlorine dioxide, comprising:
passing a gas that includes moisture to an apparatus that comprises:
a first monolith including a surface impregnated with an acid;
a second monolith including a surface impregnated with a metal chlorite; and
means for directing the gas that includes moisture sequentially to the first monolith and then to the second monolith, wherein a gaseous stream that includes moisture having passed through the first monolith carries an acid vapor desorbed from the acid surface of the first monolith to the metal chlorite of the second monolith, thereby a stream of chlorine dioxide is generated; and
directing the stream of chlorine dioxide to the enclosed space to be treated with chlorine dioxide.

64. A method of generating chlorine dioxide comprising:
passing a gas that includes moisture through a first apparatus containing a monolith material wherein the said monolith material contains a gamma alumina washcoat impregnated with an acid; and
passing the effluents containing the vapors of the said acid through a second apparatus containing a second monolith washcoated with alumina on which a metal chlorite has been impregnated, wherein the flow rate of the gas is controlled so that the concentration of chlorine dioxide produced by the reaction of the acid vapors contained in the effluent from the first monolith with the metal chlorite contained in the washcoat of the second apparatus, does not exceed substantially 15% by weight of the gas fluid.

65. A monolith for use in a canister for generating biocidal agent in a gaseous state, the canister including an inlet and an outlet, the monolith comprising:
channels for facilitating flow of a gas; and
a surface impregnated with one of an acid and a compound.

66. The monolith according to claim 65, wherein the biocidal agent is chlorine dioxide.

67. The monolith according to claim 66, wherein the compound is a metal chlorite.

68. The monolith according to claim 65, wherein the biocidal agent is ethylene oxide.

69. The monolith according to claim 68, wherein the compound is ethylene glycol.

70. The monolith according to claim 65, wherein the surface of the monolith is substantially dry.

71. The monolith according to claim 65, wherein the first monolith is made of a material chosen from the group consisting of a metal, an iron alloy, a nickel alloy, an aluminum alloy, a ceramic, and cordierite.

72. The monolith according to claim 65 wherein the acid is selected from the group consisting of sulfuric acid, hydrochloric acid, nitric acid, propionic acid, acetic acid and fluorosulphonic acid.
